(19) United States
(12) Patent Application Publication
(10) Pub. No.: US 2012/0003304 A1
(43) Pub. Date: Jan. 5, 2012

(54) Solid Forms

(75) Inventor: Yeli Zhang, Princeton, NJ (US)

(73) Assignee: FMC CORPORATION, Philadelphia, PA (US)

(21) Appl. No.: 13/170,670

(22) Filed: Jun. 28, 2011

Related U.S. Application Data
(60) Provisional application No. 61/361,091, filed on Jul. 2, 2010.

(51) Int. Cl. A61K 9/14; A61K 9/48; A61K 9/20

(52) U.S. Cl. 424/451; 424/499; 424/464

(57) ABSTRACT

The present invention is directed to a solid form comprising an active ingredient and croscarmellose, wherein: (i) the croscarmellose has a median particle size of ≧56 microns, (ii) the croscarmellose is present in an amount of ≧4% by weight based on the total weight of the solid form, and (iii) the solid form is a tablet, capsule, caplet, lozenge or granule. The present invention is also directed to a method of decreasing the disintegration time (for example, in water) of a solid form that comprises croscarmellose in an amount ≧4% by weight based on the total weight of the solid form.

### FIELD OF THE DISCLOSURE

The present invention is directed to a solid form comprising an active ingredient and croscarmellose, as well as to methods of decreasing the disintegration of time of solid forms containing high amounts of croscarmellose.

### BACKGROUND

Disintegrants are used to aid in the rapid break-up of material and are commonly used in a variety of solid forms such as tablets, capsules, caplets, etc. Solid forms have a variety of important applications including food and drinks (e.g, confectionery products, aromas, and sweeteners), detergents, dyes, sanitary products (e.g., laundry detergents and other cleaning products), agricultural products, pharmaceuticals, nutraceuticals, etc. Disintegrants assist in the rapid break-up of these solid forms so that their content is quickly released into a target media.

There are a number of factors that affect disintegration of a tablet, capsule, etc. Such factors include the ability of a disintegrant to swell and wick. The manufacturing process of a tablet (e.g., wet granulation vs. dry granulation vs. direct compression) is another factor which can affect dissolution. For example, in a direct compression process, an active pharmaceutical or nutraceutical ingredient (“API”) can be blended with a variety of excipients, subsequently lubricated and directly compressed into a tablet. A disintegrant used in this type of formulation must simply break the tablet apart to expose the API for dissolution. In a wet granulation process, the API is combined with other excipients and processed with the use of a solvent (aqueous or organic) with subsequent drying and milling to produce granules. The resulting granules are then blended with additional excipients prior to being compressed into a tablet. In a wet granulation process, the disintegrant can be added through intra-granulation and extra-granulation. Dry granulation is similar to wet granulation, except that compression and milling are used instead of solvents to make the granules. The disintegrant in dry granulation is also added through intra-granulation and extra-granulation.

Because of the increased demands for faster dissolution, there are now available “superdisintegrants” (in addition to disintegrants) such as microcrystalline cellulose, starch, pregelatinized starch, and sodium bicarbonate (in combination with citric or tartaric acids). Three major groups of superdisintegrants have been developed which disintegrate in water or aqueous fluid while producing minimal viscosity effects: (1) cross-linked modified starches, (2) cross-linked polyvinylpyrrolidone, and (3) internally cross-linked sodium carboxymethyl cellulose also known as croscarmellose.

Croscarmellose is commercially available from FMC Corporation and is sold under the name Ac-Di-Sol®. Ac-Di-Sol® has been found to accelerate disintegration by wicking, swelling, and some deformation recovery due to its fibrous structure. This functionality has translated into superior disintegration characteristics at low use levels (e.g., 2.0% or less) when compared to other superdisintegrants. However, Ac-Di-Sol® has not been promoted for use at higher use levels (e.g., at 4% or 5% or higher) because of decreased functionality at such higher use levels. As a result, there is a desire to broaden the application window for croscarmellose superdisintegrants at higher use levels.

### SUMMARY OF THE INVENTION

The present invention is directed to a solid form comprising an active ingredient and croscarmellose, wherein: (i) the croscarmellose has a median particle size of ≧56 microns, (ii) the croscarmellose is present in an amount of ≧4% by weight based on the total weight of the solid form, and (iii) the solid form is a tablet, capsule, caplet, lozenge or granule.

The present invention is also directed to a method of decreasing the disintegration time (for example, in water) of a solid form that comprises croscarmellose in an amount ≧4% by weight based on the total weight of the solid form. The method comprises the step of preparing any of the solid forms of the present invention.

### BRIEF DESCRIPTION OF THE FIGURES

FIG. 1 compares the disintegration time (seconds) versus compaction force (kN) for tablets containing 0.5% croscarmellose having varying median particle sizes of 34 microns (commercially available disintegrant), 59, microns, 72 microns and 86 microns.

FIG. 2 compares the disintegration time (seconds) versus compaction force (kN) for tablets containing 2% croscarmellose having varying median particle sizes of 34 microns (commercially available disintegrant), 59 microns, 72 microns and 86 microns.

FIG. 3 compares the disintegration time (seconds) versus compaction force (kN) for tablets containing 5% croscarmellose having varying median particle sizes of 34 microns (commercially available disintegrant), 59 microns, 72 microns, 86 microns, 201 microns, and 342 microns.

FIG. 4 compares the disintegration time (seconds) versus compaction force (kN) for tablets containing 8% croscarmellose having varying median particle sizes of 34 microns (commercially available disintegrant), 59 microns, 72 microns, 86 microns, 201 microns, and 342 microns.

### DETAILED DESCRIPTION OF THE INVENTION

As noted above, croscarmellose is commercially available from FMC Corporation and is sold under the name Ac-Di-Sol®. Ac-Di-Sol® has a mean average particle size of 25-55 microns (more typically, 30 microns to 45 microns) and has been found to have improved functionality against other commercially available superdisintegrants at low use levels of, for example, 2% or less, but has had decreased functionality at higher use levels, e.g., above 4% or 5%.

The present inventors have unexpectedly found that a solid form comprising an active ingredient and the presently claimed croscarmellose (having a median particle size ≧56 microns) at higher use levels (i.e., ≧4%) has a significant decrease in disintegration time across a wide range of tablet hardnesses when compared to commercially available Ac-Di-Sol® at similar high use levels. An added benefit of the present invention is that the larger particle size croscarmellose will be more compatible with other relatively large particle size tablet components thereby preventing the problems associated with component segregation prior to tabletting and capsule filling.

As a result, the present invention is directed to a solid form comprising an active ingredient and croscarmellose, wherein: (i) the croscarmellose has a median particle size of ≧56 microns, (ii) the croscarmellose is present in an amount of ≧4% by weight based on the total weight of the solid form, and (iii) the solid form is a tablet, capsule, caplet, lozenge or granule.

The solid form of the present invention contains croscarmellose having a median particle size of ≧56 microns, ≧60 microns, ≧65 microns, ≧70 microns, ≧72 microns, ≧80 microns, ≧85 microns, ≧86 microns, ≧90 microns, ≧100 microns, ≧150 microns, ≧200 microns, ≧201 microns, ≧250 microns, ≧300 microns, or ≧342 microns. A typical upper end of the median particle size of the invention for many applications may not exceed 500 microns, may not exceed 450 microns or may not exceed 400 microns.

The solid form of the present invention contains croscarmellose in an amount of ≧4%, ≧5%, ≧6%, ≧7%, ≧8%, ≧9%, ≧10%, ≧11%, or ≧12%, by weight based on the total weight of the solid form. A typical upper end for the amount of the croscarmellose to be used in the invention for many applications may not exceed 30%, may not exceed 25%, may not exceed 20% or may not exceed 15% by weight based on the total weight of the solid form.

The solid form can comprise any combination of median particle sizes, ranges, and use levels contained in the preceding two paragraphs.

The “solid form” of the invention is a tablet, caplet, capsule, lozenge, or granule. Typical tablet, capsule, caplet, granule, and lozenge sizes include ranges of 50 mg to 1,500 mg, including, for example, 100 mg, 200 mg, 250 mg, 300 mg, 350 mg, 400 mg, 450 mg and 500 mg.

The solid form can be used in a variety of applications and may contain an active ingredient comprising, for example, a pharmaceutical active, nutraceutical active, veterinary active, cosmetic active, agricultural active (e.g., herbicidal actives, insecticidal actives, etc.), industrial active or a food. In one aspect, the solid form is orally ingested and provides immediate release of the active ingredient in the target media (e.g., the stomach).

The solid form can be a suspension tablet. A suspension tablet refers to a tablet that readily disintegrates to form a suspension in liquid. Suspension tablets are useful for delivering a predetermined amount of an active ingredient in a drinkable form.

The croscarmellose and use levels of the present invention can be used in a variety of tabletting processes including wet granulation, direct compression, and dry granulation.

The present invention is also directed to a method of decreasing the disintegration time (for example, in water) of a solid form comprising croscarmellose that is present in an amount ≧4% by weight based on the total weight of the solid form. The method comprises the step of preparing any of the solid forms of the present invention. The solid form of the present invention disintegrates in water, for example, in less than 20 seconds.

There are no limitations with respect to the active ingredient. Examples of pharmaceutical active ingredients include, but are not limited to: analgesics: acetaminophen, aspirin, naproxen; anti-ulcer drugs: famotidine; antiseptics: ondansetron, granisetron, dolasetron, domperidone, metoclopramide; antihypertensive drugs: enalapril, losartan, candesartan, valsartan, lisinopril, ramipril, doxazosin, terazosin; antihistaminic drugs: loratadine, cetirizine; antipsychotic drugs: risperidone, olanzapine, quetiapine; antidepressants: paroxetine, fluoxetine, mirtazapine; analgesics and anti-inflammatory drugs: piroxicam; antihypercholesterolemic drugs: simvastatin, lovastatin, pravastatin; antimigraine drugs: zolmitriptan, naratriptan, rizatriptan; anti-epileptic drugs: lamotrigine; anti-Parkinson drugs: selegiline, apomorphine; anxiolytic drugs: diazepam, lorazepam, zolpidem; anti-asthma drugs: zafirlukast, montelukast; erection dysfunction agents: sildenafil; all in their free base form and in their acceptable pharmaceutical salts, hydrates, solvates or isomers. An active can also be one or more of alprazolam, prednisilone, zomitriptan, selegiline, baclofen, carbidopa, levodopa, desloratadine, aripiprazole, loratadine, or donepezil.

The solid form typically has a matrix that binds and holds the ingredients together while in the solid form. The matrix may be a water soluble or insoluble material. Non-limiting examples of matrix materials include dextrose, erythritol, fructose, isomalt, lactilol, maltilol, maltose, mannitol, sorbitol, starch, such as corn starch, potato starch, wheat starch, rice starch, partial α-starch, modified starch, partially modified starch, pregelatinized starch, partially pregelatinized starch, starch hydrolysate, polydextrose, and xylitol. The matrix can be a combination of constituents. The matrix material can comprise calcium phosphate, dibasic calcium phosphate, precipitated calcium carbonate, calcium silicate, light anhydrous silicic acid, carboxymethylcellulose, dextrin, ethylcellulose, gelatin, guar gum, hydrogenated vegetable oil, hydroxyethyl cellulose, hydroxypropyl cellulose, hydroxypropyl methylcellulose, microcrystalline cellulose, polyvinyl pyrrolidone, powdered gum arabic, glucose, magnesium aluminum silicate, maltodextrin, methylcellulose, polymethacrylates, sodium alginate, and zein. Moreover, the matrix can have functions in addition to binding. For example, the matrix can provide a sweet or refreshing taste.

The solid form can contain additives. Non-limiting examples include excipients, additional disintegrants, binders, acidulants, foaming agents, natural and artificial sweeteners, flavoring agents, lubricants, coloring agents, stabilizers, pH control agents, surfactants, etc. It is also possible that the croscarmellose used in the present invention is the only disintegrant or superdisintegrant contained in the solid form.

Non-limiting examples of lubricants include magnesium stearate, stearic acid, talc, sodium stearyl fumarate, sucrose fatty acid ester, polyethylenglycol, and waxes. Stearic acid and polyethylene glycol (M_{R}>2000) are known, relatively hydrophilic, lubricants.

Non-limiting examples of additional disintegrants include carboxymethylcellulose, calcium carboxymethylcellulose, sodium carboxymethyl starch, croscarmellose sodium (other than the croscarmellose used in the present invention), crospovidone, low-substituted hydroxypropyl cellulose, and hydroxypropyl starch.

Non-limiting examples of acidulants include citric acid, tartaric acid, malic acid, and ascorbic acid.

Non-limiting examples of the foaming agent include sodium hydrogen carbonate, and sodium carbonate.

Non-limiting examples of sweeteners include aspartame, sodium cyclamate, sodium saccharine, ammonium glycyrrhizinate, neohesperidine dihydrochalcone, alitame, neotame, sucralose, stevioside, sucrose, fructose, lactose, sorbitol, and xylitol.

Non-limiting examples of flavoring agents include flavors like menthol, mint, or fruit. Flavors such as raspberry, blackberry, cherry, black cherry, black currant, strawberry, grape, lingonberry, cantaloupe, watermelon, pear, apple, pineapple, mango, peach, apricot, plum, orange, lemon, lime, spearmint, peppermint, vanilla, and chocolate are suitable. Other flavors can include the flavor of bubblegum. The flavor compound can encompass a flavor enhancer, e.g. citric acid.

Non-limiting examples of coloring agents include food colors such as food yellow No. 5, food red No. 2 and food blue No. 2, edible lake pigments, and iron sesquioxide. Furthermore, the colorants can include pigments, natural food colors and dyes suitable for food, drug and cosmetic applications. A full recitation of all F.D. & C. colorants and their corresponding chemical structures can be found in the Kirk-Othmer Encyclopedia of Chemical Technology, 3rd Edition, in volume 5 at pages 857-884, of which text is incorporated herein by reference.

Non-limiting examples of stabilizers include disodium edetate, tocopherol, and cyclodextrin.

Non-limiting examples of pH control agents include citrate, phosphate, carbonate, tartarate, fumarate, acetate, and salts formed with an amino acid.

Non-limiting examples of surfactants include sodium laurylsulfate, polysorbate 80, polyoxyethylene(160), and polyoxypropylene(30)glycol.

The specific croscarmellose used in the present invention can be prepared by any method known in the art for selecting and obtaining a particle size of croscarmellose having the desired median particle size. For example, the croscarmellose can be prepared by sieving commercially available croscarmellose through a mesh sieve. The most common form of separation is simple screening in which the screen openings are selected to obtain the desired particle size. Typical Meshes include any one or combination of: 10, 12, 14, 16, 18, 20, 25, 30, 35, 40, 45, 50, 60, 70, 80, 100, 120, 140, 170, 200, 230, 270, and 325. Most screens are moved by vibration or regular motion to facilitate passage. Other known methods for particle size separation and collection may be used. For example, air aspiration can also be used, especially in jet mills, to remove fine particles by entrainment while retaining larger particles. Hammer, ball and rod mills frequently have screens on their discharge to retain large particles and media while passing fine particles. Centrifuges or hydroclones, which rely on differences in density and particle size, can also be used to separate materials to the desired size.

As used herein, the median particle size is the size where 50 volume percent of the particles have sizes less than the value given. The median particle size is also referred to herein as the D50.

All mathematical figures used herein have been rounded using standard convention for rounding numbers. Thus, for example, the figure of 71.585 microns is rounded to 72 microns.

The present invention is now described in more detail by reference to the following examples, but it should be understood that the invention is not construed as being limited thereto. Unless otherwise indicated herein, all parts, percents, ratios and the like are by weight.

### EXAMPLES

### Example 1

The croscarmellose used in this Example was prepared by sieving commercially available croscarmellose from Akzo Nobel through various sieves and the portion that did not pass through the sieves was retained and used in the testing herein. More specifically, croscarmellose having a D50 of 72 microns, 86 microns, 201 microns and 342 microns was obtained by sieving the croscarmellose through the following sieve sizes: 170 Mesh, 100 Mesh, 80 Mesh, and 60 Mesh, respectively. Croscarmellose having a D50 of 59 microns was obtained by sieving twice through a 100 Mesh. These samples were compared to a commercially available croscarmellose (Ac-Di-Sol®; FMC Corporation) having a D50 of 34 microns. The croscarmellose having a D50 of 34 microns is a comparative sample while the croscarmellose samples having a D50 of 59 microns, 72 microns, 86 microns, 201 microns and 342 microns are examples of croscarmellose that may be used in the present invention.

The D50 was determined by analyzing the dry powder using a Malvern Particle Size Analyzer (Mastersizer® 2000, Version 5.54, Malvern Instruments Ltd., Malvern, UK).

Model tablets were then prepared using varying amounts of the croscarmellose and their disintegration times were compared so as to demonstrate the impact of the croscarmellose containing different D50 at different use levels. Each of the tablets contained croscarmellose, mannitol and magnesium stearate. Spray-dried mannitol was obtained as Pearlitol® 200 SD (which is a direct compressible mannitol) from Roquette (Paris, France), and was used as the tablet matrix. Magnesium stearate (Mallinckrodt, Hazelwood, Mo.) was used as a lubricant. To prepare each formulation, the ingredients were weighed according to the ratios presented in the table immediately below.

| | 0.5% | 2.0% | 5.0% | 8.0% |
|---|---|---|---|---|
| | Tablets | Tablets | Tablets | Tablets |
| Croscarmellose | 0.5% | 2.0% | 5.0% | 8.0% |
| Mannitol | 98% | 96.5% | 93.5% | 90.5% |
| Magnesium | 1.5% | 1.5% | 1.5% | 1.5% |
| Stearate | | | | |

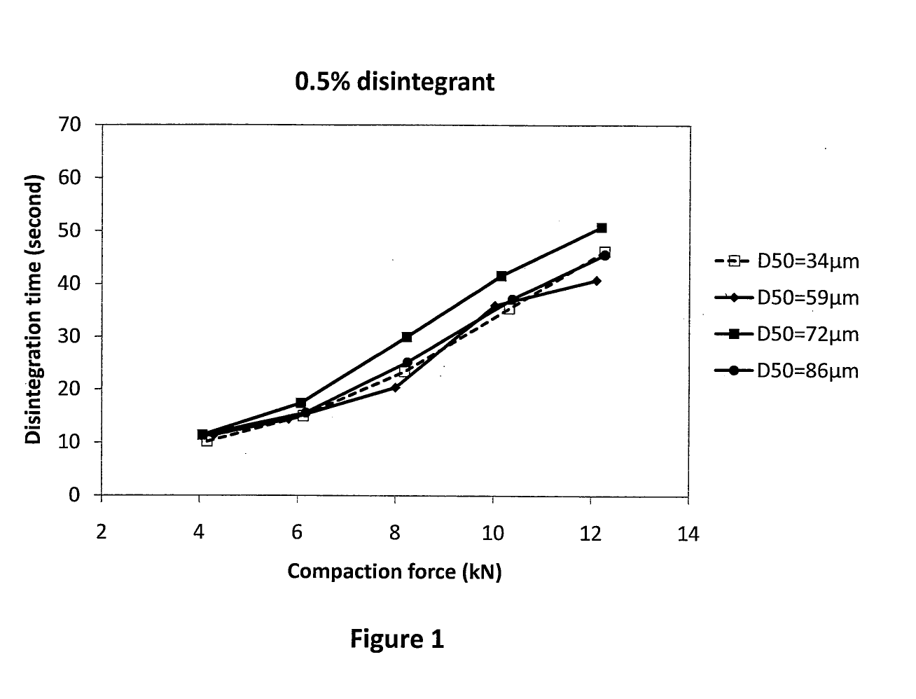

The croscarmellose and Pearlitol® 200 SD were premixed in a V-blender for 15 minutes; then magnesium stearate was added and followed up with additional 2 minutes of mixing. To prepare the tablets, each formulation was compressed individually on a Stokes 512 Tablet Press with four stations. Standard 7/16″ concave punches and corresponding dies were used. The tablet weight was adjusted to 400 mg. SMI Director™ data acquisition system was used to record the compaction process. Compaction forces of 4 kN, 6 kN, 8 kN, 10 kN, and 12 kN were applied to the formulations to produce tablets with different hardness. Disintegration of the tablets was accomplished using a DT 2-IS Disintegration Tester (Dr. Schlenniger Pharmatron). The disintegration times were determined by visually inspecting the tablets for the point in time when the tablets were fully disintegrated. The disintegration times set forth in FIGS. 1-4 are the average disintegration time for six tablets at each compaction force and use level. The test was conducted at 37±0.5 Celsius in a medium of 10 mL distilled water.

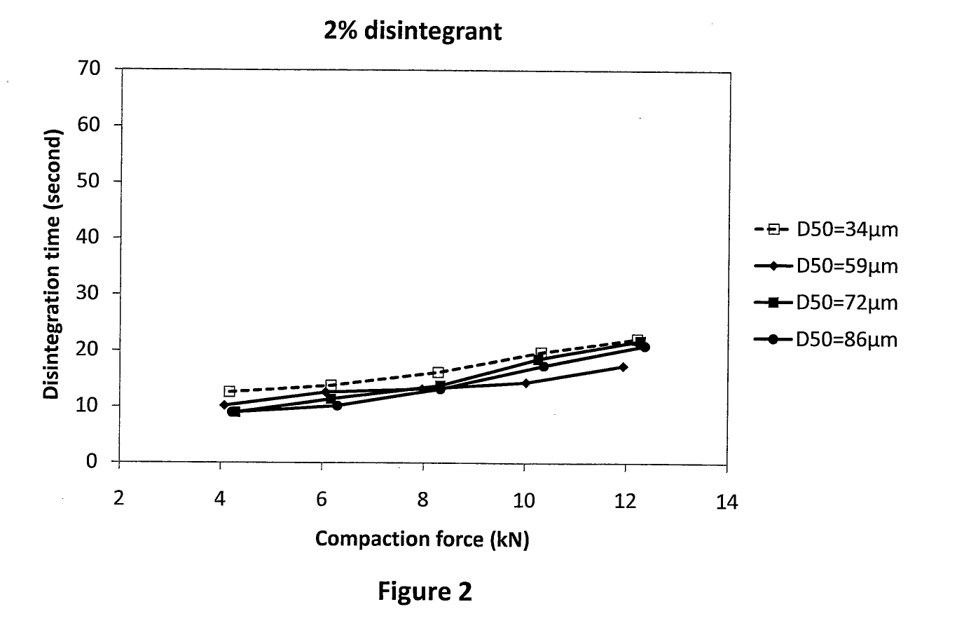

As can be seen from FIGS. 1 and 2, at 0.5% and 2% use levels, the croscarmellose having higher D50 (i.e., D50 of 59 microns, 72 microns and 86 microns) showed similar disintegration profiles at similar compaction forces as the commercially available croscarmellose (having a D50 of 34 microns). This indicates that, at relatively low use levels, the particle size of the larger croscarmellose samples did not have a significant impact on the disintegration time as compared to the commercially available sample.

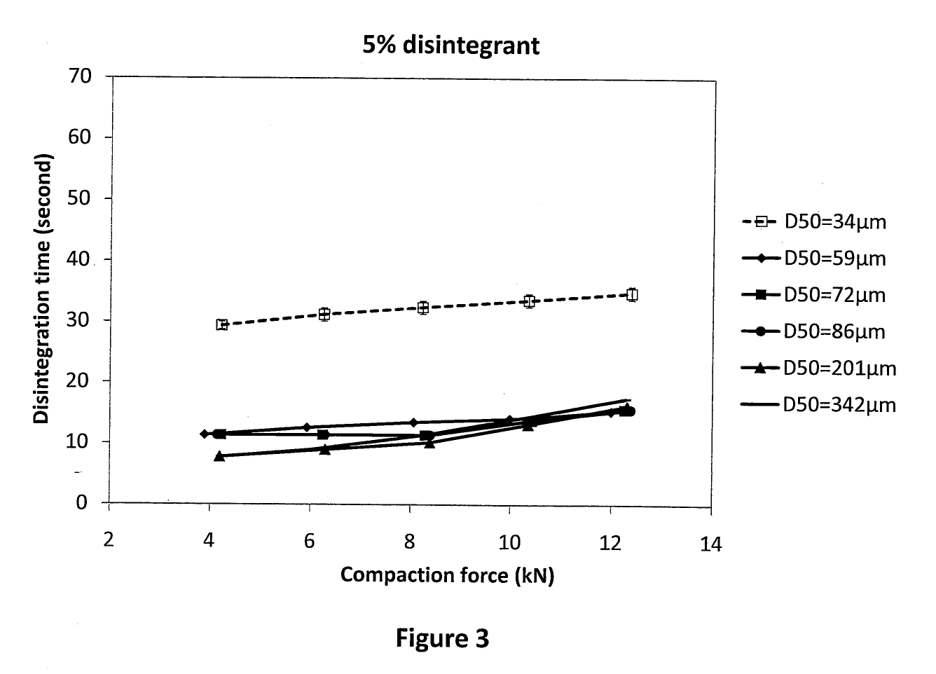

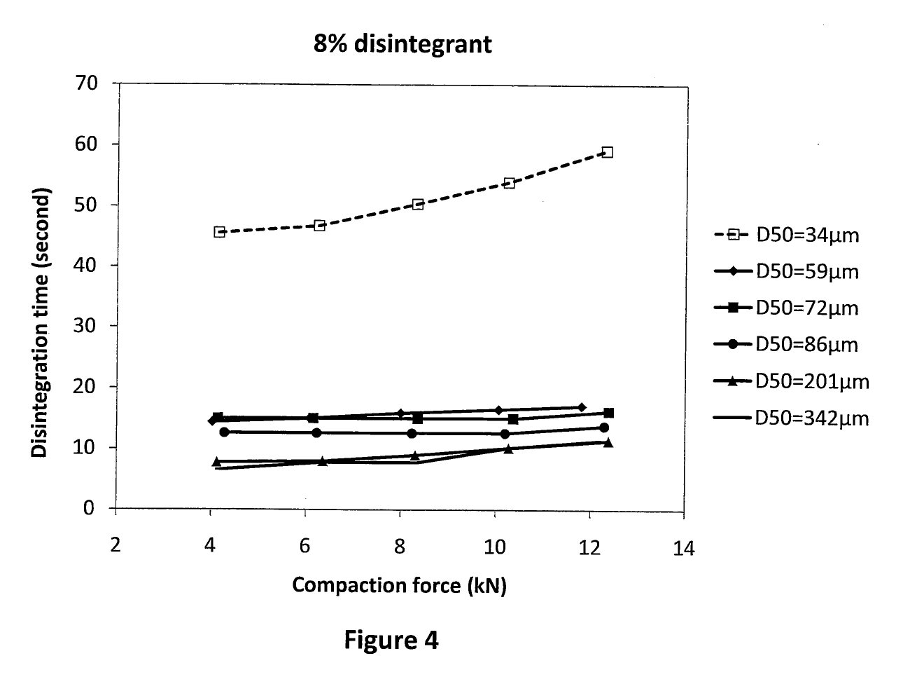

However, at the higher use levels of 5% and 8%, FIGS. 3 and 4 show at least two unexpected findings. That is, FIGS. 3 and 4 demonstrate an unexpected and significant decrease in the disintegration time for each croscarmellose sample having higher D50 (i.e., D50 of 59 microns, 72 microns, 86 microns, 201 microns and 342 microns) throughout the entire range of tested compaction forces when compared to the disintegration times at similar compaction forces for the commercially available croscarmellose (having a D50 of 34 microns). Moreover, FIGS. 3 and 4 also unexpectedly demonstrate that the difference between the disintegration times (throughout the entire tested range of compaction forces) for the croscarmellose having larger D50 (as used in the present invention) and the commercially available sample (D50 of 34 microns) actually grew as the use level increased from 5% to 8%.

While the invention has been described in detail and with reference to specific embodiments thereof, it will be apparent to one skilled in the art that various changes and modifications can be made therein without departing from the spirit and scope thereof.

It is claimed:

1. A solid form comprising an active ingredient and croscarmellose, wherein: (i) said croscarmellose has a median particle size of ≧56 microns, (ii) said croscarmellose is present in an amount ≧4% by weight based on the total weight of said solid form, and (iii) said solid form is a tablet, capsule, caplet, lozenge or granule.

2. The solid form of claim 1, wherein said active ingredient comprises at least one of a pharmaceutical active ingredient, a nutraceutical active ingredient, a veterinary active ingredient, a cosmetic active ingredient, an agricultural active ingredient, an industrial active ingredient or a food.

3. The solid form according to claim 1, wherein said croscarmellose is present in an amount of ≧5% by weight based on the total weight of said solid form.

4. The solid form according to claim 1, wherein said croscarmellose is present in an amount of ≧6% by weight based on the total weight of said solid form.

5. The solid form according to claim 1, wherein said croscarmellose is present in an amount of ≧7% by weight based on the total weight of said solid form.

6. The solid form according to claim 1, wherein said croscarmellose is present in an amount of ≧8% by weight based on the total weight of said solid form.

7. The solid form according to claim 1, wherein said croscarmellose is present in an amount of ≧9% by weight based on the total weight of said solid form.

8. The solid form according to claim 1, wherein said croscarmellose is present in an amount of ≧10% by weight based on the total weight of said solid form.

9. The solid form according to claim 1, wherein said croscarmellose is present in an amount of ≧11% by weight based on the total weight of said solid form.

10. The solid form according to claim 1, wherein said croscarmellose has a median particle size of ≧60 microns.

11. The solid form according to claim 1, wherein said croscarmellose has a median particle size of ≧65 microns.

12. The solid form according to claim 1, wherein said croscarmellose has a median particle size of ≧70 microns.

13. The solid form according to claim 1, wherein said croscarmellose has a median particle size of ≧85 microns.

14. The solid form according to claim 1, wherein said croscarmellose has a median particle size of ≧90 microns.

15. The solid form according to claim 1, wherein said croscarmellose has a median particle size of ≧200 microns.

16. The solid form according to claim 1, wherein said croscarmellose has a median particle size of ≧300 microns.

17. The solid form according to claim 1 further comprising one or more colorants, sweeteners, fragrances, flavor blockers, flavor compounds, or additional disintegrants, or a combination thereof.

18. The solid form according to claim 1, wherein said solid form is a tablet.

19. The solid form according to claim 18, wherein said solid form fully disintegrates in water in less than 20 seconds.

20. A method of decreasing the disintegration time in water of a solid form comprising croscarmellose present in an amount ≧4% by weight based on the total weight of said solid form, said method comprising the step of preparing the solid form in any one of claims 1-18.
